(19) United States
(12) Patent Application Publication
(10) Pub. No.: US 2024/0189551 A1
(43) Pub. Date: Jun. 13, 2024

(54) COATED MEDICAL GUIDEWIRES AND METHODS OF MAKING THEM

(71) Applicant: ACULON, INC., SAN DIEGO, CA (US)

(72) Inventors: Eric L. Hanson, Carlsbad, CA (US); Justin Hardin, Carlsbad, CA (US); Edward Hughes, San Diego, CA (US)

(21) Appl. No.: 18/537,706

(22) Filed: Dec. 12, 2023

Related U.S. Application Data
(60) Provisional application No. 63/432,206, filed on Dec. 13, 2022.

(51) Int. Cl. A61M 25/09; A61L 31/02; A61L 31/08; A61L 31/10

(52) U.S. Cl. CPC A61M 25/09; A61L 31/022; A61L 31/082; A61L 31/10; A61M 2025/09058; A61M 2025/09108; A61M 2025/09133; A61M 2205/0216; A61M 2205/0238

(57) ABSTRACT

Coated medical guidewires are provided comprising: (a) a guidewire substrate comprising stainless steel, surgical steel, nitinol, or CoCrMo alloy; and (b) a cured coating layer applied to the guidewire substrate (a). The cured coating layer is prepared from a curable film-forming composition comprising an aqueous solution of sodium silicate. Also provided are methods of forming coated medical guidewires comprising: (a) cleaning a guidewire substrate; (b) contacting the guidewire substrate with a curable film-forming composition comprising an aqueous solution of sodium silicate to form a coating layer on the guidewire; and (c) subjecting the guidewire substrate and film forming composition to a temperature of at least 120° C. for a time sufficient to effect cure and form a coated medical guidewire.

### CROSS REFERENCE TO RELATED APPLICATION

The present invention claims priority to U.S. Provisional Patent Application Ser. No. 63/432,206, filed Dec. 13, 2022, titled “COATED MEDICAL GUIDEWIRES AND METHODS OF MAKING THEM”, which is incorporated herein by reference in its entirety.

### FIELD OF THE INVENTION

The present invention relates to coated medical guidewires and methods of forming them.

### BACKGROUND OF THE INVENTION

A guidewire is a thin, flexible, medical wire inserted into the body to guide a larger instrument, such as a catheter, central venous line, or feeding tube. The process of catheterization was noted as early as the 18th century. The first modern applications were used as early as 1844 for the cardiac catheterization of animals, and then in 1929 on humans, when the process was tested by Dr. Werner Forssmann, a German physician. He proved to his doubting colleagues that it was possible to access the heart with a wire by carrying out the procedure on himself, and, in the process, received the 1956 Nobel Prize in Medicine for developing that technique for cardiac catheterization.

Guidewires are now commonly used in many surgical and diagnostic procedures. They allow for the deployment of tools such as cameras, lights, etc., through blood vessels or other lumens, and the placement of implants such as stents. They are predominantly used in cardiovascular, interventional radiology, and gastrointestinal applications.

Medical guidewires must be designed to demonstrate sufficient rigidity and structural integrity to carry an instrument, balanced with flexibility to navigate tortuous luminal pathways without causing injury to tissue. They must also possess sufficient torquability to allow for effective steerability. They must also demonstrate corrosion resistance, tensile strength, surface lubricity, hydrophobicity, and anti-fouling to prevent damage to blood cells and to avoid adhesion to tissue surfaces. At a distal end, which is often coiled, they may have a lubricious hydrophilic coating to reduce friction. The hydrophilic coating may be thick (several microns) to cover the gaps in the coils.

The remainder of the guidewire is often provided with a PTFE coating to reduce friction of the catheters, which are thin polymer tubes that will need to glide over it, as well as the equipment used to secure and guide the main metal wire. Such coatings are generally ˜10 microns and may be subject to flaking. If flakes delaminate in vivo they could be potentially dangerous and or life threatening.

It would be desirable to provide alternative coated medical substrates useful for surgical and diagnostic procedures that demonstrate the necessary properties for safe and effective use.

### SUMMARY OF THE INVENTION

One embodiment of the present invention provides coated medical guidewires comprising:
(a) a guidewire substrate comprising at least one of stainless steel, surgical steel, nitinol, and CoCrMo alloy; and
(b) a cured coating layer applied to the guidewire substrate (a). The cured coating layer is prepared from a curable film-forming composition comprising an aqueous solution of sodium silicate.

One embodiment of the present invention includes methods of forming coated medical guidewires comprising the steps of:
(a) cleaning a guidewire substrate;
(b) contacting the guidewire substrate with a curable film-forming composition comprising an aqueous solution of sodium silicate to form a coating layer on the guidewire substrate; and
(c) subjecting the coated guidewire substrate to a temperature of at least 120° C. for a time sufficient to effect cure and form a coated medical guidewire.

These and other advantages of the present invention will be described in the following description taken together with the attached figures in which like references represent like elements throughout.

### BRIEF DESCRIPTION OF THE FIGURES

FIG. 1 is a perspective schematic view of a coated guidewire in accordance with the present invention;

FIG. 2 is a schematic sectional view of a body portion of the coated guidewire of FIG. 1; and

FIG. 3 is a schematic sectional view of an alternative version of a body portion of the coated guidewire of FIG. 1.

### DETAILED DESCRIPTION OF THE INVENTION

Other than in any operating examples, or where otherwise indicated, all numbers expressing quantities of ingredients, reaction conditions and so forth used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used in this specification and the appended claims, the articles “a,” “an,” and “the” include plural referents unless expressly and unequivocally limited to one referent.

The various aspects and examples of the present invention as presented herein are each understood to be non-limiting with respect to the scope of the invention.

As used in the following description and claims, the following terms have the meanings indicated below:

The terms “on”, “appended to”, “affixed to”, “bonded to”, “adhered to”, or terms of like import means that the designated item, e.g., a coating, film or layer, is either directly connected to (in contact with) the object surface, or indirectly connected to the object surface, e.g., through one or more other coatings, films or layers.

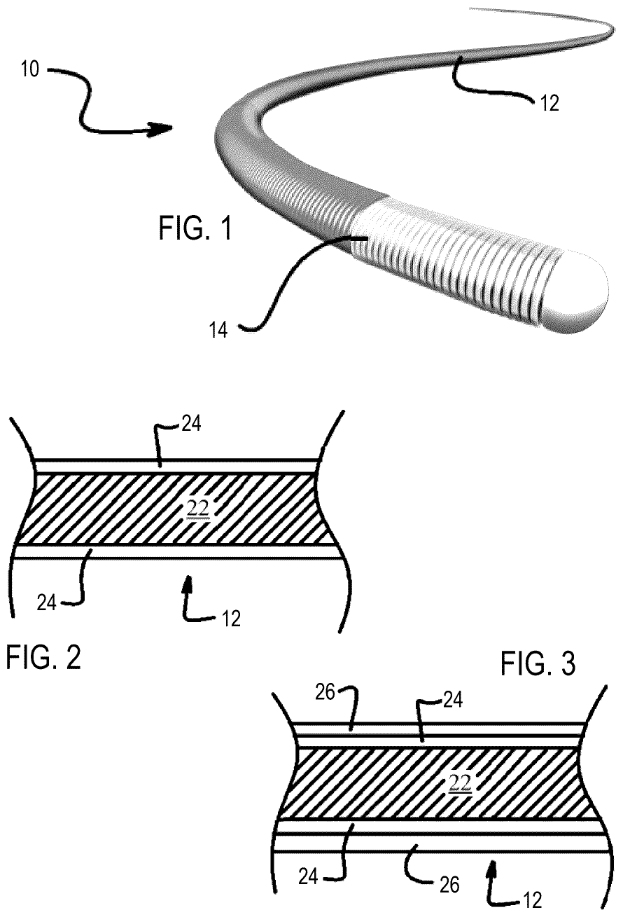

The coated medical guidewires 10 of the present invention are typically surgical or diagnostic guidewires for use in medical procedures such as cardiovascular, interventional radiology, and gastrointestinal applications. FIG. 1 is a perspective schematic view of a coated guidewire 10 in accordance with the present invention wherein the guidewire 10 can be divided into a proximal body portion 12 and a distal tip portion 14. The tip 14 is generally relatively short, being about 15 cm. FIG. 2 is a schematic sectional view of the body portion 12 of the coated guidewire 10 of FIG. 1, showing a guidewire substrate 22 and a coating 24 thereon as described in detail below. As shown in FIG. 1, the tip 14 may be in the form of a coil. The coated medical guidewires 10 comprise (a) a guidewire substrate 22 throughout the body 12 and tip 14 and (b) a cured coating layer 24 applied to the surface of the guidewire substrate 22. The guidewire substrate 22 (a) usually comprises stainless steel, surgical steel, nitinol, CoCrMo alloy, or any other material commonly used in the preparation of medical guidewires. In particular examples of the present invention, the guidewire substrate 22 (a) has a cross-sectional diameter of from 0.35 to 3.5 millimeters, depending on the application. For example, a vascular guidewire often ranges from 0.35 to 1 mm, while a guidewire designed for intestinal applications may range from 0.6 to 3.5 mm or larger.

Prior to application of any coatings, the guidewire substrate 22 may be cleaned, usually to remove contaminants such as oxide scale formed during pretreatment processes (e.g., heat treatment and stress relief processing) of the guidewire substrate 22. The guidewire substrate 22 may be cleaned by applying an aqueous alkaline solution to the guidewire such as a solution of potassium hydroxide. The solution may further comprise surfactants. This solution may demonstrate a high pH (14 and above). Application may be by brushing, wiping, immersion, or any method that allows for complete coverage of the guidewire surface, and may be followed if necessary by rinsing with deionized or distilled water. Alternatively, the guidewire substrate 22 may be cleaned by immersing the guidewire substrate 22 in an aqueous electrolytic solution and applying an appropriate voltage such as in the range from 2 to 100V to the guidewire substrate 22. Examples of suitable electrolytic solutions include mineral acid solutions such as hydrochloric acid, sulfuric acid, nitric acid, and the like. In another example, the guidewire may be cleaned by wet sanding, such as with 1500 grit SiN paper, and rinsed with deionized or distilled water.

After cleaning, the guidewire substrate is contacted with a curable film-forming composition to form a coating layer 24 on the guidewire substrate 22. The curable film-forming composition comprises an aqueous solution of sodium silicate. The sodium silicate is usually present in the curable film-forming composition in an amount of 5 to 15 percent by weight, such as 7 to 12 percent by weight, for example, 10 percent by weight, based on the total weight of the curable film-forming composition. The curable film-forming composition may additionally comprise a low surface energy fluorosurfactant, enabling wetting, spreading, leveling, and other beneficial properties. An example of a suitable surfactant is CAPSTONE FS-31, commercially available from The Chemours Company FC, LLC. Such surfactants may be present in the curable film-forming composition in an amount of 0.01 to 0.08 percent by weight, such as 0.03 to 0.06 percent by weight, for example, 0.05 percent by weight, based on the total weight of the curable film-forming composition.

Adjuvant materials may be present in the film-forming composition. Examples include solvents, viscosity (rheology) modifying components such as shear thinning or thixotropic compounds, stabilizers such as sterically hindered alcohols and acids, surfactants and anti-static agents. Exemplary organic solvents include alcohols such as methanol, ethanol and propanol, aliphatic hydrocarbons such as hexane, isooctane and decane; ethers, for example, tetrahydrofuran, and dialkylethers such as diethylether.

The adjuvants, if present, are individually present in amounts of up to 30 percent by weight based on the non-volatile (solids) content of the composition.

In certain examples of the present invention, the curable film-forming composition further comprises a polyethylene oxide polymer having siloxane functional groups. Additionally, the polyethylene oxide polymer may have other functional groups such as alkyl, carboxyl, hydroxyl, and phosphonic acid or salts thereof.

An example of a suitable polyethylene oxide polymer is bis(3-triethoxysilylpropyl)polyethylene oxide (EO=25-30 units) available from Gelest. Modification of the polyethylene oxide polymer with particular functional groups may be performed using art-recognized methods. When used, the polyethylene oxide polymer is present in the curable film-forming composition in an amount of 0.5 to 3.5 percent by weight, based on the total weight of the curable film-forming composition, such as 1 percent by weight or 3 percent by weight.

The curable film-forming composition may be applied to the guidewire substrate 22 (a) by conventional means such as dipping (immersion), spraying, or wiping to form a film. The guidewire substrate 22 is most effectively contacted with the curable film-forming composition by immersion in a vessel containing the curable film-forming composition, and the guidewire substrate 22 with the coating layer 24 formed thereon is removed from the curable film-forming composition in the vessel at a rate of up to 15 cm per minute, such as 10 cm per minute. After application of the curable film-forming composition to the guidewire substrate 22, as water evaporates from the composition, silanol groups present in the composition begin to react with each other via condensation to form a glasslike oxide coating layer 24 on the surface of the guidewire substrate 22. Exposure to CO₂ in the air continues the reaction by removing Na ions as sodium carbonate, and densifies the film.

After application of the coating layer 24, the guidewire substrate 22 (with layer 24) may be subjected to a temperature of at least 120° C. for a time sufficient to effect cure. Temperatures of 120 to 150° C. are typical, although higher temperatures are possible for stainless steel guidewires. Cure times may range from 1.5 to 2.5 hours; two hours is typically sufficient.

The term “cure”, “cured” or similar terms, as used in connection with a cured or curable composition, e.g., a “cured composition” of some specific description, means that at least a portion of any polymerizable and/or crosslinkable components that form the curable composition is polymerized and/or crosslinked. Additionally, curing of a composition refers to subjecting said composition to curing conditions such as those listed above, leading to the reaction of the reactive functional groups of the composition. The term “at least partially cured” means subjecting the composition to curing conditions, wherein reaction of at least a portion of the reactive groups of the composition occurs. The composition can also be subjected to curing conditions such that a substantially complete cure is attained and wherein further curing results in no significant further improvement in physical properties, such as hardness.

The dry film thickness of the cured coating layer 24(b) is typically 10 nm to 1 micron, and is significantly less than that of conventional PTFE coatings applied to medical guidewires.

Thus, the present invention is further drawn to a method of forming a coated medical guidewire 10, comprising the steps of:
(a) cleaning a guidewire substrate 22 as described above;
(b) contacting the guidewire substrate 22 with the curable film-forming composition as described comprising an aqueous solution of sodium silicate to form a coating layer 24 on the guidewire substrate 22; and
(c) subjecting the guidewire substrate 22 and layer 24 to a temperature of at least 120° C. for a time sufficient to effect cure and form a coated medical guidewire 10.

In certain examples of the present invention, a hydrophobic coating layer 26 may be applied to the cured coating layer 24 as shown in FIG. 3. The hydrophobic coating layer 26 is typically prepared from a composition comprising a fluoroalkylether siloxane. An example of a suitable fluoroalkylether siloxane is FSM 3750, available from Cytonix. When a hydrophobic coating layer 26 is applied to the cured coating layer 24, the curing step (c) may be shortened to about 1 hour. After application of the hydrophobic coating layer 26, curing may be continued, usually for up to one hour at a temperature of at least 120° C. as necessary.

Not intending to be bound by theory, the polyethylene oxide (PEO) additive in the curable film-forming composition that forms the coating layer 24 is expected to migrate to the surface of the silicate coating and crosslink with it during the curing process. The PEO will demonstrate similar friction profiles (when wet) to conventional PTFE coatings. The fluoroalkyl ether siloxane monolayer “topcoat” without any PEO additive is also similar in friction characteristics to PTFE coatings.

It is also possible to include the fluoroalkylether siloxane in the curable film-forming composition and apply a single coating layer 24 to the guidewire substrate 22, followed by a single curing step. In this scenario, the fluoroalkylether siloxane is present in the curable film-forming composition in an amount of usually 0.1 to 1% based on the total weight of the curable film-forming composition, but generally would not be combined with the silicate solution.

Whereas particular embodiments of this invention have been described above for purposes of illustration, it will be evident to those skilled in the art that numerous variations of the details of the present invention may be made without departing from the scope of the invention as defined in the appended claims.

What is claimed is:

1. A coated medical guidewire comprising:
(a) a guidewire substrate comprising stainless steel, surgical steel, nitinol, or CoCrMo alloy; and
(b) a cured coating layer applied to the guidewire substrate (a), wherein the cured coating layer is prepared from a curable film-forming composition comprising an aqueous solution of sodium silicate.

2. The coated medical guidewire of claim 1, wherein the guidewire substrate (a) comprises at least one of stainless steel and nitinol.

3. The coated medical guidewire of claim 1, wherein the guidewire substrate (a) demonstrates a cross-sectional diameter of from 0.35 to 3.5 mm (0.35 to 1, 0.6 to 1, depending on application).

4. The coated medical guidewire of claim 1, wherein the sodium silicate is present in the curable film-forming composition in an amount of 5 to 15 percent by weight, based on the total weight of the curable film-forming composition.

5. The coated medical guidewire of claim 1, wherein curable film-forming composition further comprises a fluorosurfactant, present in the curable film-forming composition in an amount of 0.01 to 0.08 percent by weight, based on the total weight of the curable film-forming composition.

6. The coated medical guidewire of claim 1, wherein the curable film-forming composition further comprises a polyethylene oxide polymer having siloxane functional groups, wherein the polyethylene oxide polymer is present in the curable film-forming composition in an amount of 0.5 to 3.5 percent by weight, based on the total weight of the curable film-forming composition.

7. The coated medical guidewire of claim 1, further comprising (c) a hydrophobic coating layer applied to the cured coating layer, wherein the hydrophobic coating layer is prepared from a composition comprising a fluoroalkylether siloxane.

8. The coated medical guidewire of claim 1, wherein the cured coating layer is less than 1 micron thick.

9. A method of forming a coated medical guidewire, comprising the steps of:
(a) cleaning a guidewire substrate;
(b) contacting the guidewire substrate with a curable film-forming composition comprising an aqueous solution of sodium silicate to form a coating layer on the guidewire; and
(c) subjecting the guidewire substrate and the film forming composition to a temperature of at least 120° C. for a time sufficient to effect cure and form a coated medical guidewire.

10. The method of claim 9, wherein the guidewire is cleaned in step (a) by
(i) applying an aqueous alkaline solution to the guidewire substrate and then optionally rinsing with water; or
(ii) immersing the guidewire substrate in an aqueous electrolytic solution and applying a voltage of 2-100 volts to the guidewire.

11. The method of claim 10, wherein the guidewire is cleaned in step (a) by applying the aqueous alkaline solution to the guidewire substrate, wherein the aqueous alkaline solution comprises potassium hydroxide.

12. The method of claim 10, wherein the guidewire is cleaned in step (a) by immersing the guidewire substrate in the aqueous electrolytic solution, wherein the aqueous electrolytic solution comprises a mineral acid.

13. The method of claim 12, wherein the mineral acid comprises sulfuric acid.

14. The method of claim 9, wherein the sodium silicate is present in the curable film-forming composition in an amount of 5 to 15 percent by weight, based on the total weight of the curable film-forming composition.

15. The method of claim 9, wherein curable film-forming composition further comprises a fluorosurfactant, present in the curable film-forming composition in an amount of 0.01 to 0.08 percent by weight, based on the total weight of the curable film-forming composition.

16. The method of claim 9, wherein curable film-forming composition further comprises a polyethylene oxide polymer having siloxane functional groups, wherein the polyethylene oxide polymer is present in the curable film-forming composition in an amount of 0.5 to 3.5 percent by weight, based on the total weight of the curable film-forming composition.

17. The method of claim 9, wherein the guidewire substrate is contacted with the curable film-forming composition by immersion, and the guidewire substrate with the coating layer formed thereon is removed from the curable film-forming composition at a rate of up to 15 cm per minute.

18. The method of claim 9, further comprising:
(d) applying a hydrophobic coating layer to the cured coating layer, wherein the hydrophobic coating layer is prepared from a composition comprising a fluoroalkylether siloxane.

19. The method of claim 9, wherein the guidewire is cleaned in step (a) by
(i) applying an aqueous alkaline solution to the guidewire substrate and then optionally rinsing with water, wherein the aqueous alkaline solution comprises potassium hydroxide; or
(ii) immersing the guidewire substrate in an aqueous electrolytic solution and applying a voltage of 2-100 volts to the guidewire, wherein the aqueous electrolytic solution comprises sulfuric acid; and further comprising:
(d) applying a hydrophobic coating layer to the cured coating layer, wherein the hydrophobic coating layer is prepared from a composition comprising a fluoroalkylether siloxane.

20. The guidewire formed by the method of claim 9.
